(19) United States
(12) Patent Application Publication
(10) Pub. No.: US 2008/0003590 A1
(43) Pub. Date: Jan. 3, 2008

(54) Termination of Binding Parameters

(76) Inventors: Johannes Schmitt, Leipzig (DE); Joachim Noeller, Leipzig (DE); Thomas Mueller, Eppelheim (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/573,236

(22) PCT Filed: Jul. 29, 2005
(86) PCT No.: PCT/EP05/08226

§ 371 Date: Aug. 17, 2007

(30) Foreign Application Priority Data
Aug. 5, 2004 (DE) 102004038873.3

(51) Int. Cl. C12Q 1/68; G01N 21/76; G01N 33/00; G01N 33/53; G01N 33/573

(52) U.S. Cl. 435/6; 435/7.1; 435/7.4; 436/172; 436/63; 436/86

(57) ABSTRACT

The invention relates to a method for determining binding parameters between at least one type of substance and at least one binding partner by using at least one luminescent probe and an appropriate kit. For carrying out the inventive method, a solid body which is at least partially loaded with at least one binding partner and which is incubated with the testable substance is used. After separation of the solid body and a non-bound substance, the concentration of the testable substance is determined by measuring the modulation or weakening of luminescence signals of at least one probe by the non-bound substance in a supernatant.

The present invention relates to a method for the determination of binding parameters and a kit for the implementation of this method.

The determination of binding parameters between a substance and a binding partner is of considerable importance, for example in pharmacology, medicine, biotechnology and biochemistry. It allows insights as to how molecules (for example biological molecules such as for example peptides, proteins and nucleic acids, in particular biologically active materials or active substances) interact with biological membranes, how they are transported within cells and are able to exhibit a signaling action. The knowledge of binding parameters is thus particularly advantageous for the development of therapeutic active substances and other, novel medicaments [Böhm, H. J., Klebe, G. and Kubinyi, H., “Wirkstoffdesign, Spektrum” [Active substance design and spectrum], Akademischer Verlag, 1^{st} Edition, Heidelberg (1996)].

For the determination of binding parameters it is at present as a rule necessary to separate the bound and free fractions of the test substance after the binding process. This separation step can for example be effected by the use of a semipermeable membrane (dialysis), by ultrafiltration or ultra-centrifugation. The concentration of the substance in the free fraction or in the bound fraction can then be determined. In this way, conclusions can be drawn about the binding affinity of the substance to the binding partner. A disadvantage here is that further transfer steps of the phases containing the bound or non-bound fraction are usually necessary after the separation. This does not meet the requirements for a high throughput screening method (HTS method), that should enable the testing of hundreds to thousands of substances per day. To simplify the separation of free and bound fractions of a substance, one binding partner (for example a protein or a lipid membrane) can be immobilized on a solid body. In this case the separation can be effected by sedimentation, filtration or by a simple, low-speed centrifugation. However, at least one further transfer step from liquids is also necessary here for the determination of the concentration of the substance.

In order to avoid the costly transfer of free and bound fractions of test substance after a separation, various approaches for performing a determination of binding parameters without separation of substance and binding partner with the use of so-called marker substances have already been pursued. For example, the use of fluorescent or radioactive markers which bind in specific regions of the binding partner to be tested enables the quantification of the binding of the test substances via suppression of the fluorescent [D. E. Epps, T. J. Raub, F. J. Kezdy “A General, Wide-Range Spectrofluorometric Method for Measuring the Site-Specific Affinities of Drugs toward Human Serum Albumin”, Analytical Biochemistry 227, 342-350 (1995)] or radioactive markers [N. Bosworth and P. Towers “Scintillation proximity assay” Nature 341, 167-168 (1989)]. Without the use of marker substances the determination of binding parameters can normally only be effected in special cases. Among these is the study of protein binding events wherein intrinsic, detectable properties of the protein change after the binding. An example of this is the binding of substances to human serum albumin [D. E. Epps, T. J. Raub, V. Caiolfa, A. Chlari, M. Zamal “Determination of the Affinity of Drugs toward Serum Albumin by Measurement of the Quenching of the Intrinsic Tryptophan Fluorescence of the Protein” J. Pharm. Pharmacol. 51, 41-48 (1999)]. This is however only possible in the case of very strongly binding substances and in many cases leads to error-prone results on account of the UV activity of the test substances. Such an approach is also described in the literature for the determination of the binding parameters to α1-acid glycoprotein and also to membrane proteins [A.-K. Johansen, N.-P. Willasen, G. Sager “Fluorescence studies of β-adrenergic ligand binding to α1-acid glycoprotein with 1-anilino-8-naphthalene sulfonate, isoprenaline, adrenaline and propranolol” Biochemical Pharmacology” 43(4), 725-729, (1992); R. Liu, A. Siemiarcuk, F. J. Sharom “Intrinsic fluorescence of the P-glycoprotein multidrug transporter: sensitivity of tryptophan residues to binding of drugs and nucleotides” Biochemistry 39(48): 14927-14938 (2000)]. These are also special cases, which cannot be generalized. Further possibilities consist in the modification of the binding partners themselves [J. P. Vilardaga, M. Bünemann, C. Krasel, M. Castro, M. J. Lohse “Measurement of the millisecond activation switch of G protein-coupled receptors in living cells” Nat. Biotechnol. 21(7), 807-812 (2003)], with one or several fluorescent probes, preferably in the vicinity of the binding pockets, binding wherein causes a change in the fluorescence signal. A further approach utilizes so-called surface plasmon resonance (SPR) [for example: P. Mistrik, F. Moreau, J. M. Allen “BlaCore analysis of leptin-leptin receptor interaction: evidence for 1:1 stoichiometry” Anal. Biochem. 327(2), 271-277 (2004)] to quantify the interaction between the binding partners. However, the devices currently obtainable also do not allow high sample throughput. Other methods for the determination of binding parameters utilize nuclear magnetic resonance (NMR), paramagnetic electron resonance (EPR) [E. de-Paula, S. Schreier “Molecular and physicochemical aspects of local anaesthetic-membrane interaction” Brazilian Journal of Medical and Biological Research 29, 877-894 (1996)] and differential calorimetry (DSC) [V. Plotnikov, A. Rochalski, M. Brandts, J. F. Brandts, S. Williston, V. Frasca, L. N. Lin “An autosampling differential scanning calorimeter instrument for studying molecular interactions” Assay Drug Dev Technol. 1, 83-90 (2002)]. These are indeed very precise and provide further information apart from the simple binding, however, they only enable the determination of a few substances a day.

The previous methods for the determination of binding parameters thus either necessitate laborious separation and/or transfer steps in order to effect a separation of bound and non-bound fractions of the test substance, or are only usable in certain cases. They are therefore either unsuitable or only insufficiently suitable for a generally applicable HTS procedure. Furthermore, conventional methods are generally not very user-friendly and are an obstacle to simple automation.

The invention thus addresses the problem of providing a method and a corresponding kit with which the stated disadvantages are avoided. In particular, the method should enable a generally applicable determination of binding parameters, which is not restricted to special cases. The method should be usable without great expense and be suitable for high sample throughput.

This problem is solved by a method such as is described in claim 1. The dependent claims 2 to 16 relate to preferred embodiments of the method. Claim 17 relates to a kit for the determination of binding parameters. Preferred embodiments of this kit are mentioned in the dependent claims 18 to 26. By reference, the wording of all claims is hereby made part of the description.

The method according to the invention for the determination of binding parameters between at least one substance and at least one binding partner uses at least one luminescent probe. Here luminescence is used as a generic term in particular for fluorescence and phosphorescence, but also for example for electron, thermo- and chemiluminescence. Firstly at least one solid body is prepared, which is at least partly loaded with at least one binding partner. The test substance or substances are contacted with this solid body, so that interactions between substance and binding partner, in particular binding, can arise. This incubation of the solid body with the at least one substance is followed by a separation of the solid body, which can now bear the bound substance, and a non-bound substance in a reaction vessel into sediment and supernatant. For the determination of the substance concentration in the supernatant, a modulation or attenuation of luminescence signals of the at least one probe by non-bound substance in the supernatant is measured. This requires overlapping of the absorption spectra of the at least one substance with excitation and/or emission spectra of the at least one probe.

The method according to the invention thus utilizes the so-called internal filter effect, wherein the light absorption of substances is determinable indirectly via an attenuation of luminescence intensities of suitable probes. This phenomenon is based on the fact that the intensity of light rays which are absorbed by other dissolved substances on the path to a suitable probe or on the path from the probe to a detector, in other words attenuated. The overlapping of excitation or emission wavelengths with the absorption bands of the test substance, on measurement at suitable wavelengths (in other words in the region of the absorption bands of the substance), leads to a modulation or attenuation of the luminescent intensity which is proportional to the concentration of the test substances in the supernatant. This effect is thus particularly advantageously suitable for the determination of the concentration of the test substance in the supernatant according to the method according to the invention.

The internal filter effect has already been utilized for various analytical methods. For example WO 94/17388 describes the use of the internal filter effect for the determination of ions in liquids with the aid of a sensor membrane. U.S. Pat. No. 4,822,746 relates in general to the use of the internal filter effect for the determination of ligand binding, ligands being understood here to mean ions, substances or the like. The ligand to be detected is here rendered accessible to analysis by a suitable (color) reaction. Moreover, U.S. Pat. No. 4,654,300 describes the use of such a quenching effect in immuno-assays. In each of these stated cases, the internal filter effect is utilized for analytical purposes. In no case however is this effect directly or exclusively evoked by the test substance, as is provided in the method according to the invention.

As well as the internal filter effect, the method according to the invention requires a spatial segregation of bound and free substance within a reaction vessel, wherein for this segregation a separation into sediment and supernatant is effected. The sediment comprises the solid body with binding partner and, if applicable, with bound substance. The non-bound substance is contained in the supernatant. As a result of this, a compartmentalization into a predominantly liquid phase and a largely solid phase is achieved. This segregation of bound and non-bound fractions of the test substance within one vessel requires no further transfer step for the segregation of sediment and supernatant. Rather, with the aid of the internal filter effect the concentration of the test substance in the supernatant can be determined directly in the reaction vessel.

In principle, there are two possible options for the implementation of the method according to the invention, which can if appropriate be combined with one another. In the first option, the luminescent probe is located in the sediment during the measurement. In the second option, the luminescent probe is located in the supernatant during the measurement.

According to the first option, the luminescent probe is applied onto the solid body which can be loaded with binding partner. For this, the solid body is preferably equipped with suitable probes before immobilization of the binding partner. Further, it can also be preferable that the solid body itself displays luminescent properties.

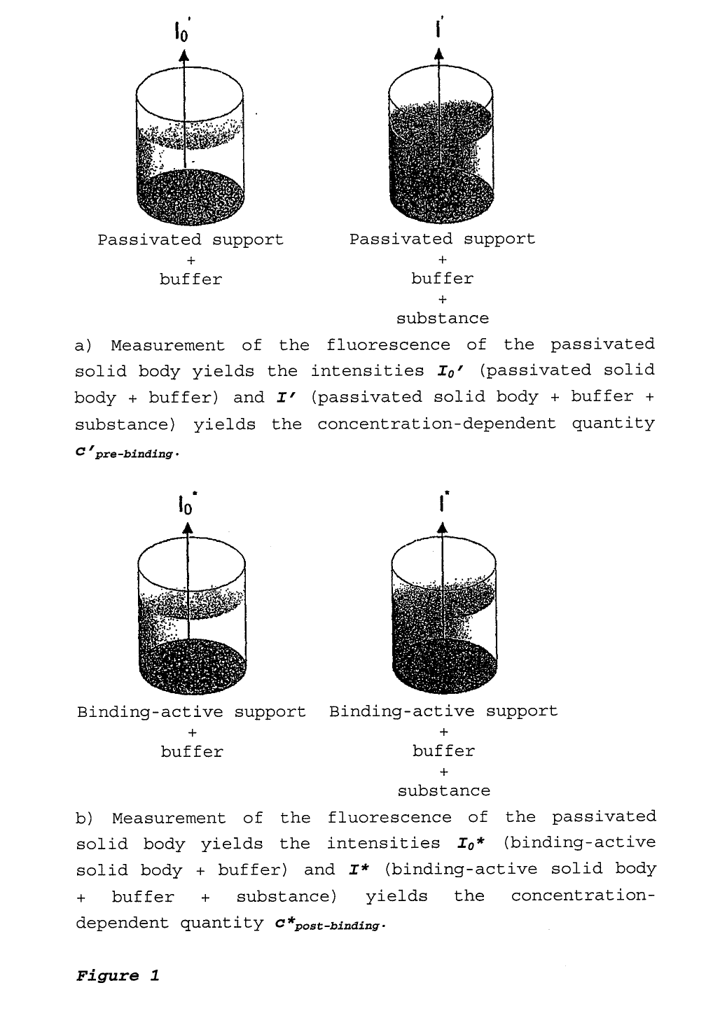

In a preferred embodiment of the method, in particular according to this option, on the one hand solid bodies as far as possible without specific and/or as far as possible without non-specific binding affinity to the test substance are provided as so-called binding-passive solid bodies and/or other solid bodies with specific binding affinity to the substance as so-called binding-active solid bodies for the method according to the invention. Here, the solid body with specific binding affinity is generally loaded with the at least one binding partner or bears the binding partner in immobilized form. The binding-passive solid bodies are as far as possible inert or inertized against non-specific binding of the substance molecules, and the inertization can be effected via a covalent and/or non-covalent saturation of reactive groups. They are as a rule used for the determination of the starting concentration, i.e. for the substance concentration before the binding to the binding partner. The binding-active solid bodies with the immobilized binding partner are used for the determination of the substance concentration after the binding. For clarification of this method, reference is made to FIG. 1.

In a preferred embodiment of this option for the method according to the invention, probe-labeled substances or solid bodies are added to the binding-active and binding-passive solid bodies, which thus no longer need necessarily to be probe-labeled. The added probe-labeled substances or solid bodies then enable a determination of the substance concentration by means of the internal filter effect. This method requires a largely equal sedimentation behavior of probe-labeled substances or solid bodies and the binding-active and binding-passive solid bodies.

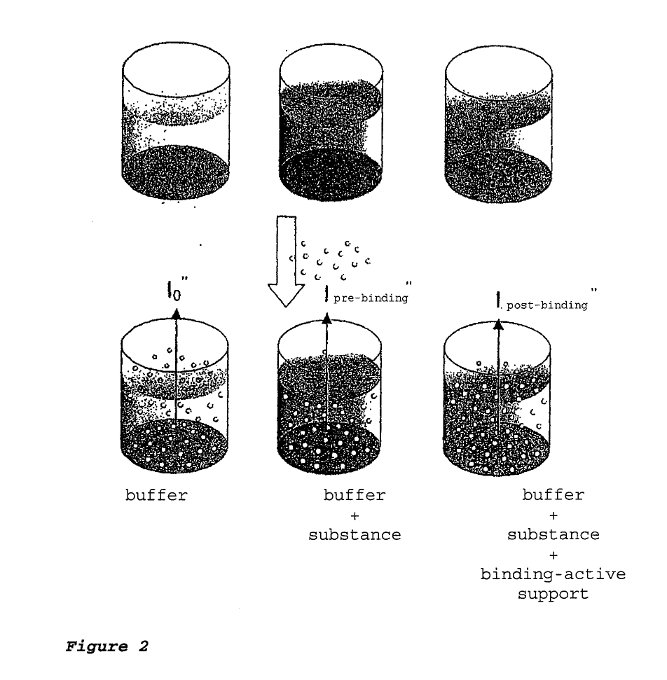

According to the second option for the method according to the invention, the probe is located in the supernatant during the measurement. Here the detection of the substance concentration is performed by means of the internal filter effect in the largely liquid phase, i.e. the supernatant, wherein luminescent probes are present in the supernatant as particles which do not sediment out. This is preferably achieved through the luminescent particles having a density comparable with the aqueous phase, i.e. for example with water or buffer. Moreover, these particles are preferably large enough to scatter ultraviolet and visible light. Thus they preferably have optical dispersive properties. Owing to these light-scattering properties of the particles or probes, the luminescent signal, in particular the excitatory light, does not reach the sedimented solid body and a uniform optical path length is established. For clarification of this method, reference is made to FIG. 2.

In a particularly preferred embodiment of this option for the method, the addition of the luminescent probes, in particular of the particles, is effected after the binding and spatial segregation or separation of the solid body. On the other hand, it is also possible to place the luminescent particles or probes together with the solid bodies and the controls in the reaction vessels beforehand, and to add the test substance at the end. This embodiment requires that the dispersive and luminescent particles remain in suspension during the sedimentation of the solid body.

The two basic options for the implementation of the method according to the invention differ mainly in the fact that in the second option the luminescent particles or probes remain in the supernatant and are not located in the sediment or the solid body phase after the separation. Moreover, with the second option the use of binding-passive solid bodies is not necessary, but is possible. However, with both options, the spatial segregation of bound and free substance fractions within the reaction space or the reaction vessel is the essential requirement for implementation of the method.

The measured luminescence signals are preferably concentration-dependent quantities, by means of which the binding is quantified. For this, the luminescent intensities of the binding-passive solid body are measured with and without substance in the supernatant. The determination of the concentration-dependent quantity derives from the Lambert-Beer Law:
c′=c′_{pre-binding}=log I′₀/I′=ε.c.d
Here, I′₀ is the luminescent intensity in the reaction vessel with buffer solution and I′ the luminescent intensity in the reaction vessel with substance solution. For the determination of the binding, the solid body is loaded with the binding partner, and the binding-active solid body is separated from the free substance fraction for example by sedimentation or centrifugation. Preferably, the measurement of a standard without substance is again performed and the concentration-dependent quantity for the substance in the supernatant is determined on the basis of the Lambert-Beer Law
c*=c*_{post-binding}=log I*₀/I*=ε.c.d
By comparison of the concentrations before and after the binding, the binding can be quantified.

In a particularly preferred embodiment, the at least one luminescent probe has fluorescent and/or phosphorescent properties. Here, either the excitation of the fluorescence or phosphorescence in question is effected at one or several defined wavelengths, or excitation spectra are recorded. Furthermore, a luminescent probe which emits a signal without prior optical excitation can also be used as the probe. For example, a probe with electro-, thermo- and/or chemi-luminescence can be used for this. However, the use of probes with fluorescence and phosphorescence is particularly preferable, since these are procedures established in laboratory practice, which are very easy to manage.

In a further preferred embodiment of the method according to the invention, a single probe or a single probe type is used. On the other hand, it can also be preferable to use a combination of different probes. Such a combination of probes can preferably be mutually coupled via so-called Förster energy transfer.

In a preferred embodiment of the method according to the invention, the solid body or bodies consist of a glass, ceramic, silicate, metal and/or polymer material or of combinations of these materials. Quite especially preferred are solid bodies in the form of spherules, so-called beads, these preferably being porous spherules. Quite particularly preferred are such spherules, preferably porous spherules, of silicate material.

In a preferred embodiment of the method according to the invention, the binding partner is at least one lipid membrane. This can be a simple lipid layer, a so-called monolayer, or a double lipid layer, a so-called bilayer. The composition of such lipid layers can be freely selected depending on the desired use. For example, such lipid layers can be essentially homogeneously structured, or for example several lipids can be used as components of the membrane layers, whereby for example the circumstances of a natural membrane can be reproduced. Moreover, it can also be preferable for the lipid membrane to be a so-called biomembrane, which is in particular produced from natural membranes. Naturally, the composition of such a biomembrane can vary. According to the method according to the invention, such lipid membranes or biomembranes are immobilized on a solid body. Here, these membranes (monolayer or biolayer) can be covalently attached. It is however particularly preferable if these lipid layers are not covalently attached, so that they reflect the natural conditions of a lipid membrane particularly well. By means of such immobilized lipid layers, the binding parameters of a test substance can particularly advantageously be analyzed with lipid layers, as is of interest for many aspects in the testing of potential active substances or the like.

In another preferred embodiment, peptides, proteins, carbohydrates, surfactants, steroids, polymers, nucleotides, oligonucleotides, DNA and/or RNA can be used according to the invention as binding partners. Particularly preferably, immobilized proteins, in particular serum proteins, for example human serum albumin, enzymes or antibodies, are used as binding partners. The binding parameters of a test substance can thereby be studied with one or several of these binding partners according to the method according to the invention.

In a further preferred embodiment, the solid body loaded with binding partner(s) can also contain other components. For example, a further protein or several proteins can be attached on or within a lipid membrane which is immobilized on the solid body. In particular, these can be proteins reconstituted in the lipid membrane. Furthermore, carbohydrates, surfactants, steroids, polymers, nucleotides, oligonucleotides, DNA and/or RNA or also peptides can also be immobilized on the solid body as further components. Particularly preferably, this is effected in combination with a lipid membrane, in particular a lipid double layer. This can for example be a reconstituted ligand, for example a protein. By integration of one or several proteins, carbohydrates or other further components within a lipid membrane, in particular a lipid double layer, natural conditions of a lipid membrane can be reproduced particularly advantageously, so that by means of such relatively complex binding partners a largely natural binding behavior of the test substance can be reproduced on the basis of a lipid membrane. On the other hand, other combinations, for example proteins and carbohydrates, can be immobilized on the solid body as binding partners.

In principle, all classes of substance are possible test substances. In particular, by means of the method according to the invention, potential active substances, in particular potential pharmaceutical and/or biological active substances, which are usable for the therapy and/or diagnosis of various diseases, can be tested. These can for example be peptides or proteins. In particular, low molecular weight substances, such as often arise in the case of potential pharmaceutical active substances, can also be tested. Moreover, potential environmental pollutants, which can have different compositions or be classifiable into different substance classes, can for example be tested.

A particular advantage of the method according to the invention consists in the fact that the separation into sediment and supernatant is effected without especial cost. By means of the separation, the bound and free fraction of the test substance are spatially segregated from one another, so that the determination of the concentration in the supernatant can be performed within the reaction vessel using the internal filter effect. It is particularly preferable that the separation be performed by sedimentation and/or centrifugation, in particular by low-speed centrifugation. Above all, a simple sedimentation has the advantage that no apparatus cost whatever is necessary for this and that the segregation into sediment and supernatant takes place with no further intervention. The performance of the separation by means of a centrifugation, in particular a low-speed centrifugation, has the advantage that the separation process can thereby be accelerated and in some cases reinforced, which can be advantageous, depending on the desired application.

The method according to the invention can be performed within normal reaction vessels. Particularly preferable here is the use of microtiter plates, in particular the use of the wells of a microtiter plate as the reaction vessel. Microtiter plates of normal dimensions are suitable for this, and for example microtiter plates with 384 depressions (wells) can be used, which firstly provide sufficient volume within the individual well and secondly enable a high sample throughput. In addition, the method according to the invention can of course also be performed in other suitable apparatus.

In a particularly preferred embodiment, the measurement of the luminescence signal is effected at one or several discrete wavelengths, in particular excitation and emission wavelengths. On the other hand, however, whole spectra can also be measured and conclusions drawn therefrom as to the modulation of the particular signals at specific wavelengths.

In a preferred embodiment, the excitation of the probes is effected at a defined wavelength by irradiation “from above”, wherein therefore the excitation takes place on the supernatant side. This does not of course necessarily have to be the upper side of the system. With appropriate configuration of the whole reaction system, for example in a centrifuge, this can also for example mean irradiation from the side. This excitatory wavelength passes through the supernatant and is thereby attenuated by light absorption by the unbound substances in the supernatant. This has the effect that only a decreased proportion of the irradiation signal impinges on the probe in the sediment or even in the supernatant, depending on the method option, and hence the luminescence signal emitted by the probe is decreased compared to corresponding controls. With the use of luminescent probes which emit a signal without prior excitation, the emitted signal is as a rule recorded on the supernatant side, since this internal filter effect phenomenon can thereby be appropriately utilized.

In a further preferred embodiment, the measurement of the emitted signal, i.e. the emitted radiation from the probe, is effected on the sediment side, i.e. without passing through the supernatant. The excitation with a suitable wavelength is also preferably effected on this side (measurement “from below”). The result of this is that an altered luminescent intensity after binding of the test substance to the binding partner in the sediment can be taken into account. This form of measurement thus serves mainly for normalization of the measurement results. This can preferably be used for a normalization of the luminescent intensity of the measurement “from above”, so that the luminescence modulation due to the substance bound to the solid body can be corrected for. Preferably, particularly in the first option for the method according to the invention, a measurement is made both “from above” and also “from below”. On the other hand, it can also be preferable if the measurement is made exclusively by measurement of the emitted radiation and preferably also excitation of the probes “from below”, for example in the determination of relatively high concentrations. In the second described option for the method according to the invention, the excitation and/or measurement of the emitted radiation preferably also is effected “from above”. For this, once again either the excitation or the emission spectra can be determined, or the luminescence signals can be determined at one or several discrete excitation and emission wavelengths. Of course, combinations of the various possibilities are also possible.

Preferably, the determination of the substance concentration in the supernatant is effected by determination of concentration-dependent quantities, as has already been described. As an option to this or in combination therewith, it can be preferable that the substance concentration be determined on the basis of calibration lines. For this, calibration lines are advantageously recorded before and/or after the binding, for which the concentration of the substance is varied in the presence of the luminescent probes, for example the particles according to the second option for the method, and the proportionality constant α is determined according to with the Lambert-Beer Law:
c″=log I″₀/I″=ε.c.d=α.c
Here, I″₀ is the luminescent intensity, in particular the fluorescence and/or phosphorescence intensity, of the buffer solution and I″ the luminescent intensity, in particular the fluorescence and/or phosphorescence intensity, of the substance solution. On the other hand, as well as this, or as an option thereto, the concentration-dependent quantities c″_{pre-binding} and c″_{post-binding} can be determined and the binding quantified by comparison of the two parameters.

The invention also includes a kit for the determination of binding parameters, which comprises at least one solid body and at least one luminescent probe. The probe preferably has fluorescent and/or phosphorescent properties and is located in the sediment or in the supernatant during the measurement according to the described method. Probes with luminescent properties without optical excitation, in particular electron, thermo- and/or chemiluminescent properties, can also be preferred.

Preferably the solid body is at least partially loaded with at least one binding partner. On the other hand, it can also be preferable that the solid body be provided in the kit according to the invention without further loading, so that the user is given the option of performing a loading with suitable binding partners himself. This results in greater flexibility in the possibilities for use of the kit according to the invention, so that the user can easily adapt the components of the kit according to the invention to the particular aims of the experiment to be performed. Above all in the case where the solid body is provided in the kit without loading with a binding partner, other components can be contained in the kit which can be used by the user for loading or immobilization with a binding partner, for example suitable buffers, linker molecules or the like.

In a preferred embodiment of the kit, the solid body is loaded with a lipid layer, preferably with a lipid double layer. Loading with a lipid monolayer can also be preferred. The immobilization is for example effected via covalent forces. Particularly preferred, however, is immobilization via non-covalent forces, for example by lipophilic interactions, in particular van der Waals interactions, and/or electrostatic interactions. This has the advantage that natural conditions of a membrane can thereby be particularly well imitated. Concerning a suitable immobilization of lipid layers, reference is made to WO 99/51984.

In a further preferred embodiment of the kit according to the invention, the solid body loaded with binding partner(s) is provided as a solid body with immobilized proteins. Particularly preferred here are serum proteins immobilized on the solid body, for example human serum albumin, enzymes or antibodies. The solid body-immobilized proteins can for example be immobilized on the solid body directly or via suitable linker molecules.

In a further preferred embodiment, the binding partner is formed of a lipid layer which contains reconstituted proteins within the layer. Hereby, for example, binding affinities between substance and binding partner can be analyzed, the binding partner in the narrower sense being the protein which is presented to the substance for binding in its natural surroundings, namely the lipid layer or a lipid double layer. The natural conditions in binding reactions can thus be particularly advantageously reproduced. The proteins which are immobilized on the solid body either on and/or within a lipid layer as binding partners are preferably membrane and/or transmembrane proteins, in particular serum proteins, preferably human serum albumin.

In a preferred embodiment of the kit according to the invention, the solid body is provided with at least one luminescent probe. This embodiment of the kit according to the invention is mainly suitable for performance of the method according to the first option described above. In another preferred embodiment of the kit according to the invention, the probe has optical dispersive properties and is not coupled with the solid body. This embodiment of the kit according to the invention is mainly suitable for performance of the method according to the invention according to the second option described above.

Particularly advantageous is the provision of the solid body loaded at least partially with at least one binding partner in prepipetted microtiter plates in for example 96, 384 and 1536 well (cavity) format, wherein the solid body-immobilized binding partner, if appropriate the probe, and the buffer are already contained, so that the user only has still to add the substance and if appropriate the probe.

Concerning further features of the various components of the kit according to the invention, reference is made to the above description.

The use of luminescence-labeled solid bodies and/or dispersive and luminescent particles according to the method according to the invention, in combination with the solid body-supported binding between an immobilized binding partner and a test substance, constitutes a generally applicable method for the determination of binding parameters. The determination of the concentration-dependent quantities can advantageously be performed directly after segregation into sediment and supernatant by simple sedimentation or the like. Herein, the test substance itself as it were serves as the marker. As a result, a concentration change after the binding is directly and exclusively detected by the test substance and is accessible as a modulation of the luminescence signal. The method according to the invention therefore particularly advantageously suits the requirements of an automated and user-friendly high-throughput screening method, such as is preferably used in the pharmaceutical industry.

The features described and further features of the invention are accessible from the following description of examples in combination with the subclaims and the diagrams. Herein, the individual features can each be implemented per se or in combination with one another.

In the diagrams

FIG. 1: shows a diagrammatic representation of the measurement principle according to the first option for the method according to the invention;

FIG. 2: shows a diagrammatic representation of the measurement principle according to the second option for the method according to the invention;

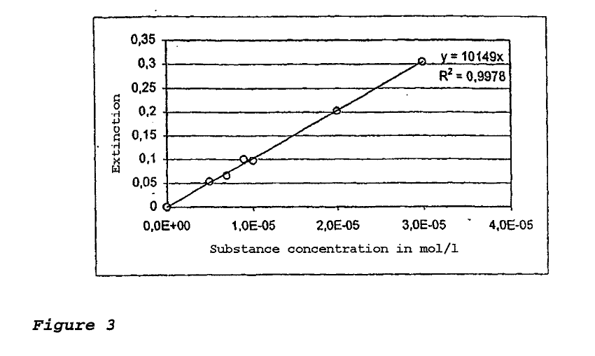

FIG. 3: shows the concentration dependence of the concentration-dependent quantity log F₀(λ)/F(λ)=ε(λ).c.d for warfarin;

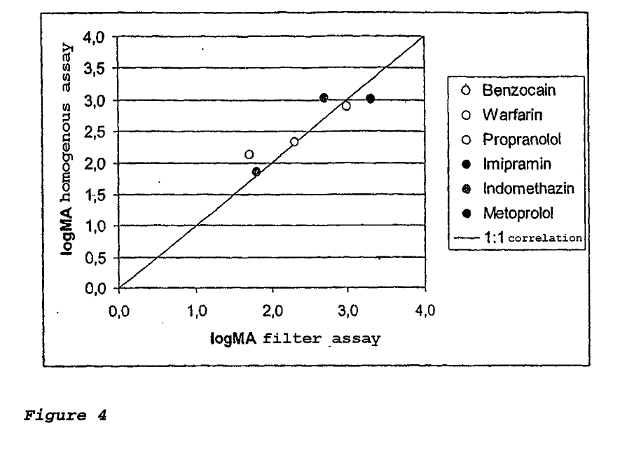

FIG. 4: shows the correlation of the membrane affinities logMA measured according to the invention with the values from conventional membrane affinity measurements using filter assays;

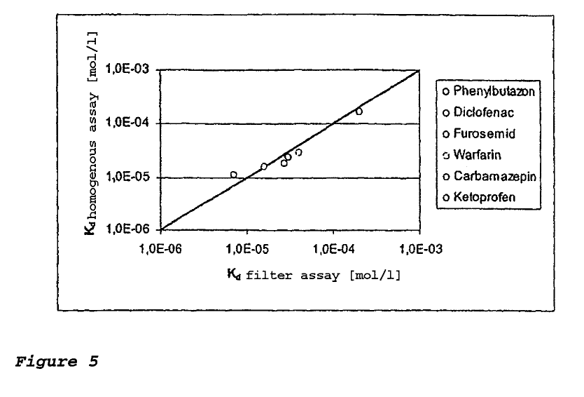

FIG. 5: shows the correlation of the K_{d} (dissociation constant) values for HSA binding with values from dissociation constants conventionally determined using filter assays;

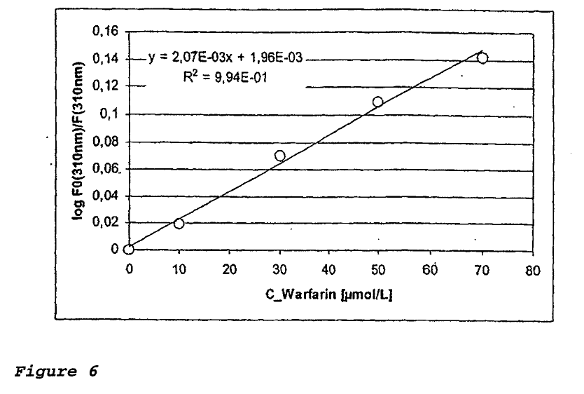

FIG. 6: shows the concentration dependence of the concentration-dependent quantity log F₀(λ)/F(λ)=ε(λ).c.d in strongly dispersive media for warfarin.

FIG. 1 shows a diagrammatic representation of the measurement principle of the method according to the invention in the form of the first option for the method in a preferred embodiment. Herein, the measurement of the fluorescence of the passivated solid bodies yields the intensities I′₀ (passivated solid body plus buffer), and I′ (passivated solid body plus buffer plus substance) yields the concentration-dependent quantity c′_{pre-binding}. The measurement of the fluorescence of the binding-active solid bodies yields the intensities I₀* (binding-active solid body plus buffer), and I* (binding-active solid body plus buffer plus substance) yields the concentration-dependent quantity c*_{post-binding}. FIG. 2 in a comparable manner shows the measurement principle of the method according to the invention according to the second option in a preferred embodiment. Herein, I₀ is the fluorescent intensity of the buffer solution, I_{pre-binding}″ the fluorescent intensity of the substance solution and I_{post-binding}″ the fluorescent intensity after the binding event and compartmentization into sediment and supernatant.

Below, the implementation of the method according to the invention is described on the basis of the determination of the affinity of warfarin to a lipid membrane, the determination of the albumin binding of carbamazepine and on the basis of the albumin binding of warfarin in strongly dispersive media.

### EXAMPLES

### 1. Preparation of Amino Supports

2 g of a porous silicate material (Nucleoprep 300-12, Macherey-Nagel, Düren) are placed in a silane solution consisting of 9.2 ml of N-(2-aminoethyl)-3-aminopropyl-trimethoxysilane and 243 μl of concentrated acetic acid in 450 ml of deionized water, and slowly rotated for three hours, after which the silicate material is sedimented, washed three times with deionized water and dried at 80° C.

### 2. Preparation of Fluorescence-Active Supports

1 mg of 3-indolylacetic acid is dissolved in 0.5 ml of dimethylformamide and 10 mg of N-hydroxysuccinimide (NHS) and 12 mg of 1-(3-dimethylaminopropyl)-3-ethyl-carbodiimide hydrochloride, also dissolved in 0.5 ml of dimethylformamide, are added to this solution. This solution is stirred for at least 2 hrs, and then 200 mg of the amino support prepared in 1. are added. This suspension is rotated overnight and washed three times with PBS (phosphate-buffered saline, 10 mM sodium phosphate, 0.9% sodium chloride, pH 7.4) and transferred into carbonate buffer.

100 μl of a solution of fluorescein isothiocyanate (FITC) in a carbonate buffer (1.33 mg/ml) are added to 100 mg of the support in 900 μl of carbonate buffer (pH 9.5) and rotated overnight. To remove unreacted FITC, this is first washed twice with carbonate buffer and then three times with PBS.

### 3. Preparation of Fluorescence-Active and Binding-Passive Supports (FI-Ref)

1 ml of a solution of polyethylene glycol bisglycidyl ether (Mn approx. 526) in PBS (3% (w/v)) are added to 100 mg of the fluorescence-active support from 2. and rotated overnight on the overhead rotator. Finally, it is washed three times with PBS.

### 4. Reconstitution of Lipid Membranes on Fluorescence-Active Supports (FI-EiPC)

For the reconstitution of lipid membranes, firstly 1 g of a support prepared according to 2. is treated with a solution of 25 mg of sodium polystyrenesulfonate in 25 ml of water in the overhead rotator and then washed three times each with water and PBS.

60 ml of a vesicle solution of 5 mg/ml EiPC (lipid extract from egg yolk) in PBS (prepared by homogenization in the Avestin Co EmulsiFlex-C5 homogenizer) are added to this support and incubated overnight. To remove excess lipid, this is washed three times with PBS and the support is then stored in PBS. The determination of the lipid concentration is effected by drying of defined volumes of suspension, detachment of the lipid from the solid body using an organic solvent, and then HPLC analysis.

### 5. Immobilization of Human Serum Albumin (HSA) on Fluorescence-Active Supports (FI-HSA)

For the immobilization of HSA, 100 mg of HSA dissolved in 10 ml of phosphate buffer (20 mM, pH 7.4) are added to 1 g of a support prepared according to 2. and rotated overhead for at least four hours. To remove free protein, this is washed with phosphate buffer, 1M NaCl in phosphate buffer and finally with PBS. The quantification of the protein immobilization is effected by supernatant analysis. The suspension is adjusted to a desired protein concentration in PBS and stored.

### 6. Determination of the Concentration-Dependent Quantity for Warfarin

200 mg of the binding-passivated and fluorescence-active support prepared according to 3. are made up to a total volume of 1 ml with PBS. 30 μl portions of this suspension are filled into different wells of a 96 well microtiter plate (Greiner UV-Star). By addition of defined volumes of warfarin and PBS, ten different warfarin concentrations between 0 and 2 E-4 mol/l are prepared in a total volume of 300 μl. After mixing of the wells by resuspension, the solid body is separated from the supernatant by sedimentation. The excitation spectra are measured in a plate reader (Tecan Safire) in “top-read” mode between 250 and 480 nm at an emission wavelength of 530 nm in all ten wells and calculated together in accordance with the Lambert-Beer law log F₀(λ)/F(λ)=ε(λ).c.d, wherein the excitation spectrum of the buffer solution corresponds to F₀(λ). A plot of the concentration-dependent quantity log F₀/F at the warfarin absorption maximum of 310 nm as a function of the concentration (FIG. 3) is linear.

### 7. Determination of the Membrane Affinity of Warfarin in the 96-Well Format

For the determination of the membrane affinity, 200 mg of the FI-EiPC support prepared in 4. is made up to a total volume of 1 ml with PBS, and similarly 200 mg of an FI-Ref prepared according to 3. is made up to 1 ml with PBS.

For the determination of the concentration-dependent quantities, the following volumes were placed in the wells of a 96 well UV plate (Greiner UV-Star):

Pre-Binding

Well reference 1: 100 μl FI-Ref suspension+200 μl PBS

Well substance 1: 100 μl FI-Ref suspension+170 μl PBS+30 μl warfarin solution;

Post-Binding

Well reference 1′: 100 μl FI-EiPC suspension+200 μl PBS

Well substance 1′: 100 μl FI-EiPC suspension+170 μl PBS+30 μl warfarin solution;
whereby the final concentration of warfarin in the wells is 70 μM. After mixing of the wells by resuspension the solid body is separated from the supernatant by sedimentation. For the determination of the concentration-dependent quantity c_{pre-binding}, excitation spectra of the wells reference 1 and substance 1 are recorded in the “top-read” mode between 250 and 480 nm with an emission wavelength of 530 nm, and calculated from c_{pre-binding}=log F₀ᵥ(λ)/Fᵥ(λ), where F₀ᵥ(λ) and Fᵥ(λ) are the excitation spectra of the wells reference 1 and substance 1. For the determination of the concentration-dependent quantity c_{post-binding}, excitation spectra of the wells reference 1′ and substance 1′ are recorded according to the above procedure and calculated from c_{post-binding}=log F₀ₙ(λ)/Fₙ(λ). Here F₀ₙ(λ) and Fₙ(λ) are given by the excitation spectra of the wells reference 1′ and substance 1′. With the lipid volume V_{lipid} and a total volume Vₜₒₜₐₗ in the wells reference 1′ and substance 1′, the membrane affinity of warfarin is obtained from:

$$log MA = log \frac{{V}_{total}}{{V}_{lipid}} \times \frac{{c}_{pre - binding} - {c}_{post - binding}}{{c}_{post - binding}}$$

From a triple determination, a value of 1.7±0.3 was obtained, which is in good agreement with values from conventional binding assays.

### 8. Determination of the Membrane Affinity in the 96-Well Format

For the determination of the membrane affinity, 200 mg of the FI-EiPC support prepared in 4. were made up to a total volume of 1 ml with PBS, and similarly 200 mg of an FI-Ref prepared according to 3. were made up to 1 ml with PBS.

For the determination of the concentration-dependent quantities, the following volumes were pipetted into the wells of a 96 well UV plate (Greiner UV-Star):

Pre-Binding

Well reference 1: 100 μl FI-Ref suspension+200 μl PBS

Well substance 1: 100 μl FI-Ref suspension+170 μl PBS+30 μl warfarin solution;

Well substance 2: 100 μl FI-Ref suspension+170 μl PBS+30 μl benzocaine acid;

Well substance 3: 100 μl FI-Ref suspension+170 μl PBS+30 μl propranolol solution;

Well substance 4: 100 μl FI-Ref suspension+170 μl PBS+30 μl imipramine solution;

Well substance 5: 100 μl FI-Ref suspension+170 μl PBS+30 μl indomethacin solution;

Well substance 6: 100 μl FI-Ref suspension+170 μl PBS+30 μl metoprolol solution;

Post-Binding

Well reference 1′: 100 μl FI-EiPC suspension+200 μl PBS

Well substance 1′: 100 μl FI-EiPC suspension+170 μl PBS+30 μl warfarin solution;

Well substance 2′: 100 μl FI-EiPC suspension+170 μl PBS+30 μl benzocaine acid;

Well substance 3′: 100 μl FI-EiPC suspension+170 μl PBS+30 μl propranolol solution;

Well substance 4′: 100 μl FI-EiPC suspension+170 μl PBS+30 μl imipramine solution;

Well substance 5′: 100 μl FI-EIPC suspension+170 μl PBS+30 μl indomethacin solution;

Well substance 6′: 100 μl FI-EiPC suspension+170 μl PBS+30 μl metoprolol solution;

whereby the substance concentration in the wells is 70 μM. After mixing of the wells by resuspension the solid body is separated from the supernatant by sedimentation. For the determination of the concentration-dependent quantity c_{pre-binding,i}, excitation spectra of the wells reference 1 and substance i are recorded both in the “top-read” mode and also in the “bottom-read” mode between 250 and 480 nm with an emission wavelength of 530 nm, and the spectra recorded “from above” are normalized by division by the corresponding spectra recorded “from below” and calculated from c_{pre-binding,i}=log F₀ᵥ(λ)/Fᵥᵢ(λ), where F₀ᵥ(λ) and Fᵥᵢ(λ) are the normalized excitation spectra (I_{above}/I_{below}) of the wells reference 1 and substance i. For the determination of the concentration-dependent quantity c_{post-binding,i}, excitation spectra of the wells 1′ and substance i′ are recorded according to the above procedure and calculated from c_{post-binding,i}=log F₀ₙ(λ)/Fₙᵢ(λ). Here F₀ₙ(λ) and Fₙᵢ(λ) are given by the normalized excitation spectra (I_{above}/I_{below}) of the wells reference′ and substance i′. With the lipid volume V_{lipid} and a total volume Vₜₒₜₐₗ in the wells reference 1′ and substance i′, the membrane affinity log MA, of the substance i is obtained from:

$$log {MA}_{i} = log \frac{{V}_{total}}{{V}_{lipid}} \times \frac{{c}_{pre - binding , i} - {c}_{post - binding , i}}{{c}_{post - binding , i}}$$

The good agreement of the log MA values with the comparison values obtained from a heterogeneous filter assay is shown in FIG. 4.

### 9. Determination of the Albumin Binding of Carbamazepine in the 384-Well Format

For the determination of the albumin binding, 200 mg of the FI-HSA support prepared in 5. were made up to a total volume of 1 ml with PBS, and similarly 200 mg of an FI-Ref prepared according to 3. were made up to 1 ml with PBS.

For the determination of the concentration-dependent quantities, the following volumes were pipetted into the wells of a 384 well UV plate (Greiner UV-plate):

Pre-Binding

Well reference 1: 35 μl FI-Ref suspension+65 μl PBS

Well substance 1: 35 μl FI-Ref suspension+55 μl PBS+10 μl carbamazepine solution;

Post-Binding

Well reference 1′: 35 μl FI-HSA suspension+65 μl PBS

Well substance 1′: 35 μl FI-HSA suspension+55 μl PBS+10 μl carbamazepine solution;

whereby the final concentration of carbamazepine in the wells is 70 μM. After mixing of the wells by resuspension the solid body is separated from the supernatant by sedimentation. For the determination of the concentration-dependent quantity c_{pre-binding}, excitation spectra of the wells reference 1 and substance 1 are recorded in the “top-read” mode between 250 and 480 nm with an emission wavelength of 530 nm, and calculated from c_{pre-binding}=log F₀ᵥ(λ)/Fᵥ(λ), where F₀ᵥ(λ) and Fᵥ(λ) are the excitation spectra of the wells reference 1 and substance 1. For the determination of the concentration-dependent quantity c_{post-binding}, excitation spectra of the wells reference 1′ and substance 1′ are recorded according to the above procedure and calculated from c_{post-binding}=log F₀ₙ(λ)/Fₙ(λ). Here F₀ₙ(λ) and Fₙ(λ) are given by the excitation spectra of the wells reference 1′ and substance 1′. With the concentration of HSA in the wells reference 1′ and substance 1′ c_{HSA}, and neglecting saturation effects, the dissociation constant K_{d} for carbamazepine is calculated from:

$${K}_{d} = \frac{{c}_{HSA} \times {c}_{post - binding}}{{c}_{pre - binding} - {c}_{post - binding}}$$

The K_{d} value of 1.7 E-4 [mol/l] agrees well with the values from conventional binding assays.

### 10. Determination of Albumin Binding in the 384 Well Format

For the determination of the albumin binding, 200 mg of the FI-HSA support prepared in 5. are made up to a total volume of 1 ml with PBS, and similarly 200 mg of an FI-Ref prepared according to 3. were made up to 1 ml with PBS.

For the determination of the concentration-dependent quantities, the following volumes were pipetted into the wells of a 384 well UV plate (Greiner UV-plate):

Pre-Binding

Well reference 1: 35 μl FI-Ref suspension+65 μl PBS

Well substance 1: 35 μl FI-Ref suspension+55 μl PBS+10 μl phenylbutazone solution;

Well substance 2: 35 μl FI-Ref suspension+55 μl PBS+10 μl carbamazepine solution;

Well substance 3: 35 μl FI-Ref suspension+55 μl PBS+10 μl diclofenac solution;

Well substance 4: 35 μl FI-Ref suspension+55 μl PBS+10 μl ketoprofen solution;

Well substance 5: 35 μl FI-Ref suspension+55 μl PBS+10 μl furosemide solution;

Well substance 6: 35 μl FI-Ref suspension+55 μl PBS+10 μl warfarin solution;

Post-Binding

Well reference 1′: 35 μl FI-HSA suspension+55 μl PBS

Well substance 1′: 35 μl FI-HSA suspension+55 μl PBS+10 μl phenylbutazone solution;

Well substance 2′: 35 μl FI-HSA suspension+55 μl PBS+10 μl carbamazepine solution;

Well substance 3′: 35 μl FI-HSA suspension+55 μl PBS+10 μl diclofenac solution;

Well substance 4′: 35 μl FI-HSA suspension+55 μl PBS+10 μl ketoprofen solution;

Well substance 5′: 35 μl FI-HSA suspension+55 μl PBS+10 μl furosemide solution;

Well substance 6′: 35 μl FI-HSA suspension+55 μl PBS+10 μl warfarin solution;

whereby the substance concentration in the wells is 70 μM. After mixing of the wells by resuspension the solid body is separated from the supernatant by sedimentation. For the determination of the concentration-dependent quantity c_{pre-binding,i}, excitation spectra of the wells reference 1 and substance i are recorded both in the “top-read” mode and also in the “bottom-read” mode between 250 and 480 nm with an emission wavelength of 530 nm, and the spectra recorded “from above” are normalized by division by the corresponding spectra recorded “from below” and calculated from c_{pre-binding,i}=log F₀ᵥ(λ)/Fᵥᵢ(λ), where F₀ᵥ(λ) and Fᵥᵢ(λ) are the normalized excitation spectra (I_{above}/I_{below}) of the wells reference 1 and substance i. For the determination of the concentration-dependent quantity c_{post-binding,i}, excitation spectra of the wells reference 1′ and substance i′ are recorded according to the above procedure and calculated from c_{post-binding,i}=log F₀ₙ(λ)/Fₙᵢ(λ). Here F₀ₙ(λ) and Fₙᵢ(λ) are given by the normalized excitation spectra (I_{above}/I_{below}) of the wells reference 1′ and substance i′. With the concentration of HSA in the wells reference 1′ and substance i′ c_{HSA}, and neglecting saturation effects, the dissociation constants K_{d,i} of the substances i are obtained from:

$${K}_{d , i} = \frac{{c}_{HSA} \times {c}_{post - binding , i}}{{c}_{pre - binding , i} - {c}_{post - binding , i}}$$

The good agreement of the K_{d} values with the comparison values obtained from a heterogeneous filter assay is shown in FIG. 5.

### 11. Determination of the Concentration-Dependent Quantity in Dispersive Media

10 μl portions of a suspension of europium-labeled polystyrene microparticles (diameter 1 μm, Molecular Probes, Eugene, Oreg., USA) were placed into five different wells on a 384 well microtiter plate. By the addition of defined volumes of warfarin and PBS, five different warfarin concentrations between 0 and 7E-5 mol/l were prepared in a total volume of 100 μl. The final concentration of polystyrene particles is 0.05%. The excitation spectra are measured in a plate reader (Tecan Safire) in “top-read” mode between 230 and 580 nm at an emission wavelength of 620 nm (lag time 100 μsecs, integration time 400 μsecs) and calculated together from the Lambert-Beer law log F₀(λ)/F(λ)=ε(λ).c.d, wherein the excitation spectrum of the buffer solution corresponds to F₀(λ). A plot of the concentration-dependent quantity log F₀/F at the warfarin absorption maximum of 310 nm as a function of the concentration (FIG. 6) is linear.

### 12. Determination of the Albumin Binding of Warfarin in Strongly Dispersive Media

For the determination of the albumin binding in strongly dispersive media, three wells of a 384 well microtiter plate containing commercially available TRANSIL®-HSA (Nimbus Biotechnology GmbH, Leipzig) were filled as follows:

Well buffer 1: 90 μl PBS

Well reference 1: 80 μl PBS+10 μl warfarin solution

Well substance 1: 35 μl TRANSIL®-HSA suspension+45 μl PBS+10 μl warfarin solution;

wherein the warfarin concentration in wells reference 1 and substance 1 is 70 μM. After thorough mixing and sedimentation of the solid body, 10 μl of a suspension of europium-labeled polystyrene microparticles (diameter 1 μm, Molecular Probes, Eugene, Oreg., USA) are added. The excitation spectra are measured in a plate reader (Tecan Safire) in “top-read” mode between 230 and 580 nm at an emission wavelength of 620 nm (lag time 100 μsecs, integration time 400 μsecs) in all three wells and calculated together from the Lambert-Beer law log F₀(λ)/F(λ)=ε(λ).c.d, wherein the excitation spectrum of the buffer solution corresponds to F₀(λ). Using the calibration lines recorded in 11, the concentrations in the substance and reference wells are determined, and K_{d}, determined from:

$${K}_{d , i} = \frac{{c}_{HSA} \times {c}_{substance 1}}{{c}_{reference 1} - {c}_{substance 1}}$$

was K_{d}=1.7E-5 [mol/l], which is in good agreement with the results from conventional methods.

1. A method for the determination of binding parameters between at least one substance and at least one binding partner with the use of at least one luminescent probe, comprising the process steps:
provision of at least one solid body, which is at least partially loaded with at least one binding partner,
incubation of the solid body with the at least one substance,
separation of solid body and unbound substance into sediment and supernatant in a reaction vessel and
measurement of a modulation of luminescent signals of the at least one probe due to unbound substance in the supernatant for the determination of the substance concentration in the supernatant, wherein absorption spectra of the at least one substance overlap with excitation and/or emission spectra of the at least one probe.

2. The method as claimed in claim 1, characterized in that the probe is located in the sediment during the measurement.

3. The method as claimed in claim 1, characterized in that the probe is applied onto solid body loaded with binding partner and/or onto unloaded solid body.

4. The method as claimed in claim 1, characterized in that the solid body itself has luminescent properties.

5. The method as claimed in claim 1, characterized in that the probe is located in the supernatant during the measurement, and the probe preferably has optical dispersive properties.

6. The method as claimed in claim 1, characterized in that solid bodies essentially without specific and/or essentially without non-specific binding affinity to the substance are provided as binding-passive solid bodies and/or solid bodies with specific binding affinity to the substance as binding-active solid bodies.

7. The method as claimed in claim 1, characterized in that the luminescent probe has fluorescent and/or phosphorescent properties.

8. The method as claimed in claim 1, characterized in that a single probe or a combination of probes is used.

9. The method as claimed in claim 1, characterized in that the solid body is a glass, ceramic, silicate, metal and/or polymer material, in particular in the form of spherules, preferably porous spherules.

10. The method as claimed in claim 1, characterized in that the binding partner is at least one lipid membrane, biomembrane, peptide, protein, enzyme, carbohydrate, surfactant, steroid, polymer, nucleotide, oligonucleotide, DNA and/or RNA.

11. The method as claimed in claim 1, characterized in that the separation is effected by sedimentation and/or centrifugation, in particular low-speed centrifugation.

12. The method as claimed in claim 1, characterized in that the reaction vessel is the well of a microtiter plate.

13. The method as claimed claim 1, characterized in that the luminescence signal is measured at one or several discrete wavelengths and/or as a spectrum.

14. The method as claimed in claim 1, characterized in that for the measurement the luminescence signals are recorded after passing through the supernatant.

15. The method as claimed in claim 1, characterized in that for the measurement, in particular for the normalization of the measurement, the luminescence signals are recorded without passing through the supernatant.

16. The method as claimed in claim 1, characterized in that the determination of the substance concentration in the supernatant is effected by determination of concentration-dependent quantities and/or using calibration lines.

17. A kit for the determination of binding parameters by a process as claimed in claim 1, at least comprising the following components:
at least one solid body and
at least one luminescent probe.

18. The kit as claimed in claim 17, characterized in that the solid body is loaded with at least one binding partner.

19. The kit as claimed in claim 18, characterized in that the solid body loaded with binding partner is a solid body with an immobilized lipid layer, preferably lipid double layer, in particular with a non-covalently immobilized lipid layer.

20. The kit as claimed in claim 18, characterized in that the solid body loaded with binding partner is a solid body with immobilized proteins.

21. The kit as claimed in claim 18, characterized in that the solid body loaded with binding partner is a solid body with proteins reconstituted in a lipid layer.

22. The kit as claimed in claim 20, characterized in that the proteins are at least one serum protein, in particular human serum albumin.

23. The kit as claimed in claim 17, characterized in that the solid body is equipped with the probe.

24. The kit as claimed in claim 17, characterized in that the probe has optical dispersive properties.

25. The kit as claimed in claim 17, characterized in that solid body, in particular solid body with immobilized binding partner, and preferably buffer is contained in prepipetted microtiter plates, in particular in a 96, 384 and/or 1536 well (cavity) format.

26. The kit as claimed in claim 17, characterized in that the solid body itself has luminescent properties.
